SEQUENCE LISTING (4 sequences)

SEQ ID NO: 1 - DNA, 1011 bp, Arabidopsis thaliana
atgtcgaccgctcctctctccggcttctttctcacttcactttctccttctcaatcttctctccagaaactctctcttcgtacttcttccaccgtcgcttgcctcccacccgcctcttcttcttcctcatcttcctcctcctcgtcttcccgttccgttccaacgcttatccgtaacgagccagtttttgccgctcctgctcctatcattgccccttattggagtgaagagatgggaaccgaagcatacgacgaggctattgaagctctcaagaagcttctcatcgagaaggaagagctaaagacggttgcagcggcaaaggtggagcagatcacagcggctcttcagacaggtacttcatccgacaagaaagctttcgaccccgtcgaaaccattaagcagggcttcatcaaattcaagaaggagaaatacgaaaccaaccctgctttgtacggtgagctcgcaaagggtcaaagtcctaagtacatggtgtttgcttgttcagactcacgtgtgtgtccatcacacgttctggactttcagccaggagatgccttcgtggtccgtaacatagccaacatggttcctcctttcgacaaggtcaaatacggtggcgttggagcagccattgaatacgcggtcttacaccttaaggtggagaacattgtggtgataggacacagtgcatgtggtgggatcaaagggcttatgtctttccccttagatggaaacaactccactgacttcatagaggactgggtcaaaatctgtttaccagccaagtcaaaggttatatcagaacttggagattcagcctttgaagatcaatgtggccgatgtgaaagggaggcggtgaatgtttcactagcaaacctattgacatatccatttgtgagagaaggacttgtgaagggaacacttgctttgaagggaggctactatgacttcgtcaagggtgcttttgagctttggggacttgaatttggcctctccgaaactagctctgtatga

SEQ ID NO: 2 - protein, 336 aa, Arabidopsis thaliana
MSTAPLSGFFLTSLSPSQSSLQKLSLRTSSTVACLPPASSSSSSSSSSSSRSVPTLIRNEPVFAAPAPIIAPYWSEEMGTEAYDEAIEALKKLLIEKEELKTVAAAKVEQITAALQTGTSSDKKAFDPVETIKQGFIKFKKEKYETNPALYGELAKGQSPKYMVFACSDSRVCPSHVLDFQPGDAFVVRNIANMVPPFDKVKYGGVGAAIEYAVLHLKVENIVVIGHSACGGIKGLMSFPLDGNNSTDFIEDWVKICLPAKSKVISELGDSAFEDQCGRCEREAVNVSLANLLTYPFVREGLVKGTLALKGGYYDFVKGAFELWGLEFGLSETSSV

SEQ ID NO: 3 - DNA, 780 bp, Arabidopsis thaliana
atgggaaacgaatcatatgaagacgccatcgaagctctcaagaagcttctcattgagaaggatgatctgaaggatgtagctgcggccaaggtgaagaagatcacggcggagcttcaggcagcctcgtcatcggacagcaaatcttttgatcccgtcgaacgaattaaggaaggcttcgtcaccttcaagaaggagaaatacgagaccaatcctgctttgtatggtgagctcgccaaaggtcaaagcccaaagtacatggtgtttgcttgttcggactcacgagtgtgcccatcacacgtactagacttccatcctggagatgccttcgtggttcgtaatatcgccaatatggttcctccttttgacaaggtcaaatatgcaggagttggagccgccattgaatacgctgtcttgcaccttaaggtggaaaacattgtggtgatagggcacagtgcatgtggtggcatcaaggggcttatgtcatttcctcttgacggaaacaactctactgacttcatagaggattgggtcaaaatctgtttaccagcaaagtcaaaagttttggcagaaagtgaaagttcagcatttgaagaccaatgtggccgatgcgaaagggaggcagtgaatgtgtcactagcaaacctattgacatatccatttgtgagagaaggagttgtgaaaggaacacttgctttgaagggaggctactatgactttgttaatggctcctttgagctttgggagctccagtttggaatttcccccgttcattctatatga

SEQ ID NO: 4 - protein, 259 aa, Arabidopsis thaliana
MGNESYEDAIEALKKLLIEKDDLKDVAAAKVKKITAELQAASSSDSKSFDPVERIKEGFVTFKKEKYETNPALYGELAKGQSPKYMVFACSDSRVCPSHVLDFHPGDAFVVRNIANMVPPFDKVKYAGVGAAIEYAVLHLKVENIVVIGHSACGGIKGLMSFPLDGNNSTDFIEDWVKICLPAKSKVLAESESSAFEDQCGRCEREAVNVSLANLLTYPFVREGVVKGTLALKGGYYDFVNGSFELWELQFGISPVHSI

(19) United States
(12) Patent Application Publication
(10) Pub. No.: US 2004/0248152 A1
(43) Pub. Date: Dec. 9, 2004

(54) Methods for the identification of inhibitors of carbonic anhydrase expression or activity in plants

(76) Inventors: Keith Davis, Durham, NC (US); Adel Zayed, Durham, NC (US); Robert Ascenzi, Cary, NC (US); Monica Smith, Somerville, MA (US); Betsy S. Kurnik, Wake Forest, NC (US); Douglas Boyes, Chapel Hill, NC (US); Rao Mulpuri, Apex, NC (US); Neil Hoffman, Bethesda, MD (US); Susanne Kjemtrup, Chapel Hill, NC (US); Carol Hamilton, Apex, NC (US); Jeffrey Woessner, Hillsborough, NC (US); Jorn Gorlach, Manchester, NJ (US)

Correspondence Address: Icoria, Inc., 108 T.W. ALEXANDER DRIVE, P O BOX 14528, RTP, NC, 27709-4528, US

(21) Appl. No.: 10/739,607

(22) Filed: Dec. 18, 2003

Related U.S. Application Data
(60) Provisional application No. 60/435,797, filed on Dec. 19, 2002.

(51) Int. Cl. C12Q 1/68; C12Q 1/48

(52) U.S. Cl. 435/6; 435/15

(57) ABSTRACT

The present inventors have discovered that Carbonic Anhydrase (CA) is essential for plant growth. Specifically, the inhibition of CA gene expression in plant seedlings results in reduced growth, and chlorosis. Thus, CA can be used as a target for the identification of herbicides. Accordingly, the present invention provides methods for the identification of compounds that inhibit CA expression or activity, comprising: contacting a compound with an CA and detecting the presence and/or absence of binding between said compound and said an CA, or detecting a change in CA expression or activity. The methods of the invention are useful for the identification of herbicides.

### RELATED APPLICATIONS

This application claims the benefit of U.S. Provisional Application No. 60/435,797 filed Dec. 19, 2002, herein incorporated in its entirety by reference.

### FIELD OF THE INVENTION

The invention relates generally to plant molecular biology. In particular, the invention relates to methods for the identification of herbicides.

### BACKGROUND OF THE INVENTION

Carbonic Anhydrase (CA) catalyzes the reversible hydration of CO₂ to bicarbonate and is one of the most abundant soluble proteins in the leaves of C3 higher plants, representing up to 1 to 2% of the soluble leaf protein (Reed and Graham (1981) 7 Progress in Phytochemistry, Reinhold, Harbome, and Swain, eds., Pergamon Press, Oxford, UK, pp. 47-94.) Most localization studies indicate that CA is found in the chloroplasts of C3 plants and primarily within the cytosol of mesophyll cells of C4 species. However, there have been reports of cytosolic localization in C3 plants (Kachru and Anderson (1974) 118 Planta 235-40, and Reed and Graham (1981), supra.) Within the C3 chloroplast it has been postulated that CA activity could maintain the supply of CO₂ for Rubisco by speeding the dehydration of HCO₃— by facilitating the diffusion of CO₂ across the chloroplast envelope via maintenance of the equilibrium between the inorganic carbon species (Reed and Graham (1981), supra.) In C4 plants, the cytosolic CA catalizes the hydration of CO₂ to bicarbonate, the substrate of PEPcase (Hatch and Bumell (1990) 93 Plant Physiol. 825-8.) The potential role of a cytosolic CA in C3 plants is not well established. Although enzyme activity data suggesting the presence of CA isoforms have been shown for a few species (Kachru and Anderson (1974), and Reed and Graham (1981), supra), there are no protein or DNA sequences reported for any plant cytosolic DNA. Fett and Coleman (1994) 105 Plant Physiol. 707-13, identified and characterized two Arabidopsis thaliana CA cDNA clones, one of which is an extrachloroplastic, and presumably cytosolic, isoform. Fett and Coleman have also shown that the two isoforms are differentially regulated by light and that one of them requires leaf and/or chloroplast development to be expressed.

To date there do not appear to be any publications describing lethal effects of over-expression, antisense expression or knock-out of CA in plants. Thus, the prior art has not suggested that CA is essential for plant growth and development. The present invention provides carbonic anhydrase as a target for evaluating plant growth regulators, especially herbicide compounds, including, but not limited to, determinations in C3 and/or C4 plants.

### SUMMARY OF THE INVENTION

The present inventors have discovered that antisense expression of a CA cDNA in Arabidopsis causes chlorosis and reduced growth. Thus, the present inventors have discovered that CA is essential for normal seed development and growth, and can be used as a target for the identification of herbicides. Accordingly, the present invention provides methods for the identification of compounds that inhibit CA expression or activity, comprising: contacting a candidate compound with a CA and detecting the presence or absence of binding between said compound and said CA, or detecting a change in CA expression or activity. The methods of the invention are useful for the identification of herbicides.

### BRIEF DESCRIPTION OF THE FIGURE

FIG. 1. Schematic diagram of the reversible hydration of CO₂ to bicarbonate catalyzed by the enzyme Carbonic Anhydrase (CA).

FIG. 2. Plot of percent inhibition of CA-dansylamide complex formation (y-axis) versus concentration of sulfonamide-based inhibitor (x-axis). The reaction was conducted in 50 m Tris HCl buffer, pH 8.5, containing 50 μM ZnCl₂, 0.025% Tween 20, at a dansylamide concentration of 15 μM and CA enzyme concentration of 0.05 μg/ml.

### DETAILED DESCRIPTION OF THE INVENTION

Definitions

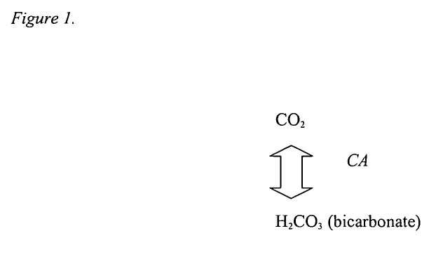

As used herein, the term “Carbonic Anhydrase” is synonymous with “CA” and refers to an enzyme that catalyzes the reversible hydration of CO₂ to bicarbonate, as shown in FIG. 1, and as included herein as the protein of SEQ ID NO: 2 and/or its encoding cDNA, SEQ ID NO: 1, and also included herein as the protein of SEQ ID NO: 4 and/or its encoding cDNA, SEQ ID NO: 3.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

As used herein, the term “cDNA” means complementary deoxyribonucleic acid.

As used herein, the term “DNA” means deoxyribonucleic acid.

As used herein, the term “dI” means deionized.

As used herein, the term “ELISA” means enzyme-linked immunosorbent assay.

As used herein, the term “GUS” means β-glucouronidase.

The term “herbicide”, as used herein, refers to a compound that may be used to kill or suppress the growth of at least one plant, plant cell, plant tissue or seed.

As used herein, the term “HPLC” means high-pressure liquid chromatography.

The term “inhibitor”, as used herein, refers to a chemical substance that inactivates the enzymatic activity of CA. The inhibitor may function by interacting directly with the enzyme, a cofactor of the enzyme, the substrate of the enzyme, or any combination thereof.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

As used herein, the term “LB” means Luria-Bertani media.

As used herein, the term “mRNA” means messenger ribonucleic acid.

As used herein, the term “Ni” refers to nickel.

As used herein, the term “Ni-NTA” refers to nickel sepharose.

As used herein, the term “PCR” means polymerase chain reaction.

The “percent (%) sequence identity” between two polynucleotide or two polypeptide sequences can be determined according to the either the BLAST program (Basic Local Alignment Search Tool, Altschul and Gish (1996) 266 Meth. Enzymol. 460-80; Altschul (1990) 215 J. Mol. Biol. 403-10) in the Wisconsin Genetics Software Package (Devererreux et al. (1984) 12 Nucl. Acid Res. 387), Genetics Computer Group (GCG), Madison, Wis. (NCBI, Version 2.0.11, default settings) or using Smith Waterman Alignment (Smith and Waterman (1981) 2 Adv. Appl. Math. 482) as incorporated into GENEMATCHER PLUS (Paracel, Inc., using the default settings and the version current at the time of filing). It is understood that for the purposes of determining sequence identity when comparing a DNA sequence to an RNA sequence, a thymine nucleotide is equivalent to an uracil nucleotide.

As used herein, the term “PGI” means plant growth inhibition.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

As used herein, the term “RNA” means ribonucleic acid.

As used herein, the term “SDS” means sodium dodecyl sulfate.

As used herein, the term “SDS-PAGE” means sodium dodecyl sulfate-polyacrylimide gel electrophoresis.

The term “specific binding” refers to an interaction between CA and a molecule or compound, wherein the interaction is dependent upon the primary amino acid sequence or the conformation of CA.

As used herein, the term “TATA box” refers to a sequence of nucleotides that serves as the main recognition site for the attachment of RNA polymerase in the promoter region of eukaryotic genes. Located at around 25 nucleotides before the start of transcription, it consists of the seven-base consensus sequence TATAAAA, and is analogous to the Pribnow box in prokaryotic promoters.

As used herein, the term “TLC” means thin layer chromatography.

Embodiments of the Invention

The present inventors have discovered that inhibition of CA gene expression strongly inhibits the growth and development of plant seedlings. Thus, the inventors are the first to demonstrate that CA is a target for herbicides.

Accordingly, the invention provides methods for identifying compounds that inhibit CA gene expression or activity. Such methods include ligand binding assays, assays for enzyme activity and assays for CA gene expression. Any compound that is a ligand for CA, other than its substrate, CO₂, may have herbicidal activity. For the purposes of the invention, “ligand” refers to a molecule that will bind to a site on a polypeptide. The compounds identified by the methods of the invention are useful as herbicides.

Thus, in one embodiment, the invention provides a method for identifying a compound as a candidate for a herbicide, comprising:

a) contacting a CA with a compound; and

b) detecting the presence and/or absence of binding between said compound and said CA,

wherein binding indicates that said compound is a candidate for a herbicide.

By “CA” is meant any enzyme that catalyzes the reversible hydration of CO₂ to bicarbonate. The CA may have the amino acid sequence of a naturally occurring CA found in a plant, animal or microorganism, or may have an amino acid sequence derived from a naturally occurring sequence. Preferably the CA is a plant CA. One cDNA (SEQ ID NO: 1) encoding the CA protein or polypeptide (SEQ ID NO: 2) can be found herein as well as in the TIGR database at locus At3g01500. Another cDNA (SEQ ID NO: 3) encoding the CA protein or polypeptide (SEQ ID NO: 4) can be found herein as well as in the TIGR database at locus At5g14740.

By “plant CA” is meant an enzyme that can be found in at least one plant, and which catalyzes the reversible hydration of CO₂ to bicarbonate. The CA may be from any plant, including monocots, dicots, C3 plants, and/or C4 plants.

In one embodiment, the CA is an Arabidopsis CA. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis CA is from Arabidopsis thaliana.

In various embodiments, the CA can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of a CA polypeptide may be used in the methods of the invention. The fragments comprise at least 10 consecutive amino acids of a CA. Preferably, the fragment comprises at least 15, 20, 25, 30, 35, 40, 50, 60, 70, 80, 90 or at least 100 consecutive amino acids residues of a CA. In one embodiment, the fragment is from an Arabidopsis CA. Preferably, the fragment contains an amino acid sequence conserved among plant carbonic anhydrases. Those skilled in the art could identify additional conserved fragments using sequence comparison software.

Polypeptides having at least 80% sequence identity with a plant CA are also useful in the methods of the invention. Preferably, the sequence identity is at least 85%, more preferably the identity is at least 90%, most preferably the sequence identity is at least 95% or 99%.

In addition, it is preferred that the polypeptide has at least 50% of the activity of a plant CA. More preferably, the polypeptide has at least 60%, at least 70%, at least 80% or at least 90% of the activity of a plant CA. Most preferably, the polypeptide has at least 50%, at least 60%, at least 70%, at least 80%, or at least 90% of the activity of an A. thaliana CA protein.

Thus, in another embodiment, the invention provides a method for identifying a compound as a candidate for a herbicide, comprising:

a) contacting a compound with at least one polypeptide selected from the group consisting of:

i) the polypeptide set forth in SEQ ID NO:2 or SEQ ID NO:4; and

ii) a polypeptide having at least 80% sequence identity with the polypeptide set forth in SEQ ID NO:2 or SEQ ID NO:4; and

b) detecting the presence and/or absence of binding between said compound and said polypeptide, wherein binding indicates that said compound is a candidate for a herbicide.

Any technique for detecting the binding of a ligand to its target may be used in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with an CA protein or a fragment or variant thereof, the unbound protein is removed and the bound CA is detected. In a preferred embodiment, bound CA is detected using a labeled binding partner, such as a labeled antibody. In a variation of this assay, CA is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for a herbicide, it can be tested for the ability to inhibit CA enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with said herbicide candidate and detecting a change in the growth or viability of said plant or plant cells. In one instance, the change detected may be a decrease in growth or viability of said plant or plant cells.

A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a candidate compound may have herbicidal activity only for certain plants or certain plant species.

The ability of a compound to inhibit CA activity can be detected using in vitro enzymatic assays in which the disappearance of a substrate or the appearance of a product is directly or indirectly detected. CA catalyzes the catalyzes the reversible hydration of CO₂ to bicarbonate. Methods for detection of CO₂ and bicarbonate include spectrophotometry, mass spectroscopy, thin layer chromatography (TLC) and reverse phase HPLC.

Thus, the invention provides a method for identifying a compound as a candidate for a herbicide, comprising:

a) contacting a CO₂ with CA;

b) contacting CO₂ with CA and a candidate compound; and

c) determining the concentration of CO₂ and/or bicarbonate after the contacting of steps (a) and (b).

If a candidate compound inhibits CA activity, a higher concentration of the substrate (CO₂) and a lower level of the product (bicarbonate) will be detected in the presence of the candidate compound (step b) than that detected in the absence of the compound (step a).

Preferably the CA is a plant CA. Enzymatically active fragments of a plant CA are also useful in the methods of the invention. For example, a polypeptide comprising at least 100 consecutive amino acid residues of a plant CA may be used in the methods of the invention. In addition, a polypeptide having at least 80%, 85%, 90%, 95%, 98% or at least 99% sequence identity with a plant CA may be used in the methods of the invention. Preferably, the polypeptide has at least 80% sequence identity with a plant CA and at least 50%, 75%, 90% or at least 95% of the activity thereof.

Thus, the invention provides a method for identifying a compound as a candidate for a herbicide, comprising:

a) contacting CO₂ with a polypeptide selected from the group consisting of:

i) the polypeptide set forth in SEQ ID NO:2 or SEQ ID NO:4; and

ii) a polypeptide having at least 80% sequence identity with the polypeptide set forth in SEQ ID NO:2 or SEQ ID NO:4; and

b) contacting said CO₂ with said polypeptide and a compound; and

c) determining the concentration of CO₂ and/or bicarbonate after the contacting of steps (a) and (b).

Again, if a candidate compound inhibits CA activity, a higher concentration of the substrate (CO₂) and a lower level of the product (bicarbonate) will be detected in the presence of the candidate compound (step b) than that detected in the absence of the compound (step a).

For the in vitro enzymatic assays, CA protein and derivatives thereof may be purified from a plant or may be recombinantly produced in and purified from a plant, bacteria, or eukaryotic cell culture. Preferably CA proteins are produced using a baculovirus or E. coli expression system. Methods for purifying CA may be found in Johansson and Forsmann (1992) FEBS Lett. 314: 232-36. Other methods for the purification of CA proteins and polypeptides are known to those skilled in the art.

As an alternative to in vitro assays, the invention also provides plant and plant cell based assays. In one embodiment, the invention provides a method for identifying a compound as a candidate for a herbicide, comprising:

a) measuring the expression of CA in a plant or plant cell in the absence of a compound;

b) contacting a plant or plant cell with said compound and measuring the expression of CA in said plant or plant cell; and

c) comparing the expression of CA in steps (a) and (b).

A change in CA expression indicates that the compound is a herbicide candidate. In one embodiment, the plant or plant cell is an Arabidopsis thaliana plant or plant cell.

Expression of CA can be measured by detecting the CA primary transcript or mRNA, CA polypeptide or CA enzymatic activity. Methods for detecting the expression of RNA and proteins are known to those skilled in the art. (See, for example, Current Protocols in Molecular Biology, Ausubel et al., eds., Greene Publishing and Wiley-Interscience, New York, 1995). However, the method of detection is not critical to the invention. Methods for detecting CA RNA include, but are not limited to, amplification assays such as quantitative PCR, and/or hybridization assays such as Northern analysis, dot blots, slot blots, in-situ hybridization, transcriptional fusions using an CA promoter fused to a reporter gene, bDNA assays, and microarray assays.

Methods for detecting protein expression include, but are not limited to, immunodetection methods such as Western blots, His Tag and ELISA assays, polyacrylamide gel electrophoresis, mass spectroscopy, and enzymatic assays. Also, any reporter gene system may be used to detect CA protein expression. For detection using gene reporter systems, a polynucleotide encoding a reporter protein is fused in frame with CA, so as to produce a chimeric polypeptide. Methods for using reporter systems are known to those skilled in the art. Examples of reporter genes include, but are not limited to, chloramphenicol acetyltransferase (Gorman et al. (1982) 2 Mol. Cell. Biol. 1104; Prost et al. (1986) Gene 45: 107-111), β-galactosidase (Nolan et al. (1988) 85 Proc. Natl. Acad. Sci. USA 2603-7), alkaline phosphatase (Berger et al. (1988) 66 Gene 10), luciferase (De Wet et al. (1987) 7 Mol. Cell Biol. 725-37), β-glucuronidase (GUS), fluorescent proteins, chromogenic proteins and the like. Methods for detecting CA activity are described above.

Chemicals, compounds, or compositions identified by the above methods as modulators of CA expression or activity can be used to control plant growth. For example, compounds that inhibit plant growth can be applied to a plant or expressed in a plant to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants are grown in Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds are surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds are sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ inch down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of 100 μ-E m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### Example 1

### Construction of a Transgenic Plant expressing the Driver

The “Driver” is an artificial transcription factor comprising a chimera of the DNA-binding domain of the yeast GAL4 protein (amino acid residues 1-147) fused to two tandem activation domains of herpes simplex virus protein VP 16 (amino acid residues 413-490). Schwechheimer et al. (1998) 36 Plant Mol. Biol. 195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### Example 2

### Construction of Antisense Expression Cassettes in a Binary Vector

A fragment or variant of an Arabidopsis thaliana cDNA corresponding to SEQ ID NO:1 was ligated into the PacI/AscI sites of an E. coli/Agrobacterium binary vector in the antisense orientation. This placed transcription of the antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E. coli. Kanamycin resistant clones were selected and purified. DNA was isolated from each clone and characterized by PCR and sequence analysis. The DNA was inserted in a vector that expresses the A. thaliana antisense RNA, which is complementary to a portion of the DNA of SEQ ID NO: 1. This antisense RNA is complementary to the cDNA sequence found in the TIGR database at locus At3g01500. The coding sequence for this locus is shown as SEQ ID NO: 1. The protein encoded by these mRNAs is shown as SEQ ID NO: 2. The same procedure could be followed for the cDNA sequence found in SEQ ID NO: 3, which encodes the protein found herein in SEQ ID NO: 4.

The antisense expression cassette and a constitutive chemical resistance expression cassette are located between right and left T-DNA borders. Thus, the antisense expression cassettes can be transferred into a recipient plant cell by agroinfection.

### Example 3

### Transformation of Agrobacterium with the Antisense Expression Cassette

The vector was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### Example 4

### Construction of an Arabidopsis Antisense Target Plants

The antisense expression cassette was introduced into Arabidopsis thaliana wild-type plants by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 25011/L Silwet L-_{77™ (}84% polyalkyleneoxide modified heptamethyltrisiloxane and 16% allyloxypolyethyleneglycol methyl ether), and transferred to a one-liter beaker.

The previously clipped Arabidopsis plants were dipped into the Agrobacterium suspension so that all above ground parts were immersed and agitated gently for 10 seconds. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial.

One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represented stably transformed T1 plants.

### Example 5

### Effect of Antisense Expression in Arabidopsis Seedlings

The T1 antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. The antisense expression of the CA gene in three separate lines resulted in significantly impaired growth, indicating that this gene represents an essential gene for normal plant growth and development. Four of nine plants from the first transgenic line, four of seven plants from the second transgenic line, and two of seven plants from the third transgenic line showed reduced growth and chlorosis. Thus, each of the three transgenic lines containing the antisense construct for carbonic anhydrase exhibited significant seedling abnormalities.

### Example 6

### Cloning and Expression Strategies, Extraction and Purification of the CA Protein

The following protocol may be employed to obtain the purified CA protein.

Cloning and expression strategies:

A CA gene can be cloned into E. coli (pET vectors-Novagen), Baculovirus (Pharmingen) and Yeast (Invitrogen) expression vectors containing His/fusion protein tags, and the expression of recombinant protein can be evaluated by SDS-PAGE and Western blot analysis.

Extraction:

Extract recombinant protein from 250 ml cell pellet in 3 mL of extraction buffer by sonicating 6 times, with 6 sec pulses at 4° C. Centrifuge extract at 15000×g for 10 min and collect supernatant. Assess biological activity of the recombinant protein by activity assay.

Isolation:

Isolate recombinant protein by Ni-NTA affinity chromatography (Qiagen).

Isolation protocol: perform all steps at 4° C.:

Use 3 ml Ni-beads (Qiagen)

Equilibrate column with the buffer

Load protein extract

Wash with the equilibration buffer

Elute bound protein with 0.5 M imidazole

In addition, the following method may be used to purify CA protein:

cDNA encoding the peaCA precursor was previously isolated and cloned into a mutagenesis/expression vector giving the plasmid pPCAt (Johansson and Forsmann (1992), 314 FEBS Lett. 232-36) The T7 RNA polymerase promoter was placed in front of the peaCA insert. An NcoI site was introduced at the initial ATG codon and a unique HindIII site was placed downstream form the stop codon. This plasmid was used to make deletion constructs by introducing additional NcoI sites using site-directed mutagenesis (Kunkei (1995) 82 Proc. Natl. Acad. Sci., USA 488-92) followed by digestion with NcoI and religation. Purification of peaCA from the E. coli strain BL2 over-expressing peaCA has been described in Johanson and Forsman (1992), supra.

For in vitro transcription the plasmids were linearised with HindIII and then transcribed using T7 RNA polymerase (Epicentre Technologies) in the presence of the cap analogue diguanosine triphosphate (Pharmacia) according to the manufacturers instructions. In vitro translations were performed in a wheat germ extract (Promega) containing 21 g mRNa and 25 μCi [³H] leucine (Amersham; specific activity 147 ci/mmol) in a total volume of 100 ul. Reaction mixtures were incubated for 60 min at 27° C.

### Example 7

### Screening Assays for Inhibitors of CA Activity

The enzymatic activity of CA may be determined in the presence and absence of candidate inhibitors in a suitable reaction mixture, such as described by the following known assay protocol:

Intact chloroplasts are isolated from 10 to 11 day old seedlings. Chlorophyll is assayed according to Bruinsma (1961) 52 Biochim. Biophys. Acta. 576-8. Eppendorf cups used for the import experiments are precoated with bovine serum albumin. The import buffer is composed of 50 mM HEPES/KOH, pH 8.0, 330 mM sorbitol, 2 mM MgCl₂, 0.5 mM dithiothreitol, 200 μg/ml antipain and 2 mM ATP. Precursors are added last before the chloroplasts (30% g chlorophyll per 150 μl import reaction). Samples are incubated for 20 min at 26° C. in the light. For the analysis of leaf extracts, plants are grown with a 17h day/7h night cycle at 26° C./15° C. and harvested after 6-24 days. The tissue is ground with an ice-cold mortar and pestle in 50 mM Tris-SO₄, pH 8.0, 10 mM DTT using 2 ml of medium/g fresh tissue, and then centrifuged at 20,000×g for 10 min at 4° C. The supernatant is analysed by SDS-PAGE and immunoblotting using anti-peaCA antiserum from rabbits and peroxidase-conjugated goat anti-rabbit IgG (BiORad).

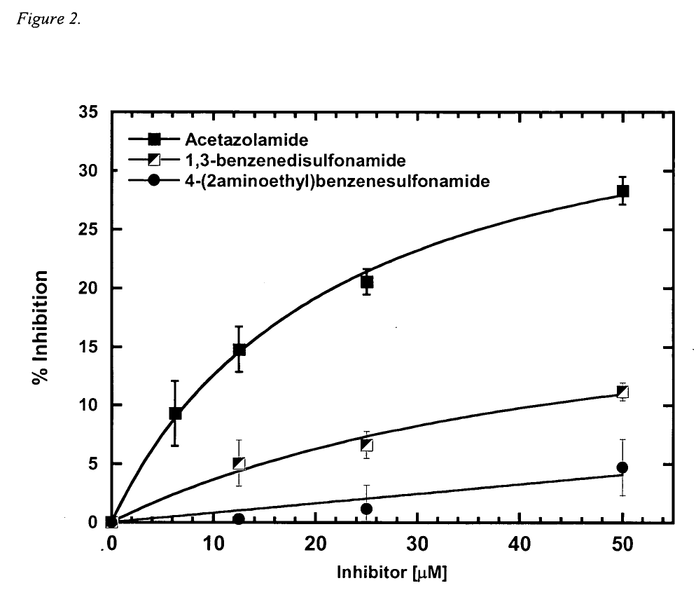

Another assay for identification of CA inhibitors is a competition-binding assay based on the binding of dansylamide to the “substrate binding pocket” of a CA enzyme. The dansylamide anion coordinates with the CA zinc (replacing the hydroxide anion) as the fourth ligand. The dansylamide-CA interaction causes a blue shift from 526 nm (free dansylamide) to 468 nm (bound dansylamide) and the appearance of a strong peak at 280 nm in the excitation spectrum of the dansylamide-CA complex. The degree of association between dansylamide and the CA enzyme can be measured from fluorescence at 460 nm (Husic and Hsieh (1992) 32 Pytochemistry 805-10). In a competition assay, inhibitors displace bound dansylamide causing a decrease in fluorescence at 460 nm. The following is an example of a CA competition binding assay procedure, for which the results are displayed in FIG. 2:

Mix 0.1 mg/ml CA solution in binding buffer (50 m Tris HCl buffer, pH 8.5, containing 50 μM ZnCl₂, 0.025% Tween 20) with an equal volume of 30 μM dansylamide solution in the same binding buffer containing an inhibitor at a concentration between 3 and 100 μM. (Inhibitors used in this instance are acetazolamide, 1,3-benzenedisulfonamide, and 4-(2-aminoethyl)benzenesulfonamide).

Incubate the mixture at room temperature for 20 min.

Measure fluorescence at 460 nm using 280 nm as excitation wavelength.

Calculate the percent inhibition of dansylamide binding from the degree of fluorescence quenching.

While the foregoing describes certain embodiments of the invention, it will be understood by those skilled in the art that variations and modifications may be made and still fall within the scope of the invention.

What is claimed is:

1. A method for identifying a compound as a candidate for a herbicide, comprising:
a) contacting a carbonic anhydrase (CA) with a compound; and
b) detecting the presence and/or absence of binding between said compound and said CA, wherein binding indicates that said compound is a candidate for a herbicide.

2. The method of claim 1, wherein said CA is a plant CA.

3. The method of claim 2, wherein said CA is an Arabidopsis CA.

4. The method of claim 3, wherein said CA is SEQ ID NO: 2 or SEQ ID NO:

5. A method for determining whether a compound identified as a herbicide candidate by the method of claim 1 has herbicidal activity, comprising: contacting a plant or plant cells with said herbicide candidate and detecting a change in growth or viability of said plant or plant cells.

6. A method for identifying a compound as a candidate for a herbicide, comprising:
a) selecting a compound that binds to a polypeptide selected from the group consisting of:
i) the polypeptide set forth in SEQ ID NO:2 or SEQ ID NO:4; and
ii) a polypeptide having at least 80% sequence identity with the polypeptide set forth in SEQ ID NO:2 or SEQ ID NO:4; and
b) contacting a plant with said compound to confirm herbicidal activity.

7. A method for determining whether a compound identified as a herbicide candidate by the method of claim 6 has herbicidal activity, comprising: contacting a plant or plant cells with said herbicide candidate and detecting a change in growth or viability of said plant or plant cells.

8. A method for identifying a test compound as a candidate for a herbicide, comprising:
a) contacting CO₂ with carbonic anhydrase (CA);
b) contacting said CO₂ with CA and a test compound; and
c) determining the concentration of at least one of CO₂ and/or bicarbonate after the contacting of steps (a) and (b), wherein a higher concentration of a substrate (CO₂) and/or a lower level of a product (bicarbonate) detected in the presence of the candidate compound (step b) than that detected in the absence of the compound (step a) indicates that said compound is a candidate for a herbicide.

9. The method of claim 8, wherein said CA is a plant CA.

10. The method of claim 9, wherein said CA is an Arabidopsis CA.

11. The method of claim 10, wherein said CA is SEQ ID NO: 2 or SEQ ID NO: 4.

12. A method for identifying a compound as a candidate for a herbicide, comprising:
a) contacting CO₂ with a polypeptide selected from the group consisting of:
i) the polypeptide set forth in SEQ ID NO:2 or SEQ ID NO: 4; and
ii) a polypeptide having at least 80% sequence identity with the polypeptide set forth in SEQ ID NO:2 or SEQ ID NO: 4; and
b) contacting CO₂ with said polypeptide and a compound; and
c) determining the concentration of at least one of CO₂ and/or bicarbonate after the contacting of steps (a) and (b) wherein a higher concentration of a substrate (CO₂) and/or a lower level of a product (bicarbonate) detected in the presence of the candidate compound (step b) than that detected in the absence of the compound (step a) indicates that said compound is a candidate for a herbicide.

13. A method for identifying a compound as a candidate for a herbicide, comprising:
a) measuring the expression of a carbonic anhydrase (CA) in a plant or plant cell in the absence of a compound;
b) contacting a plant or plant cell with said compound and measuring the expression of CA in said plant or plant cell; and
c) comparing the expression of CA in steps (a) and (b), wherein a change in CA expression between step (a) and step (b) indicates that said compound is a candidate for a herbicide.

14. The method of claim 13 wherein said plant or plant cell is an Arabidopsis plant or plant cell.

15. The method of claim 14, wherein said CA is SEQ ID NO: 2 or SEQ ID NO: 4.

16. The method of claim 13, wherein the expression of carbonic anhydrase (CA) is measured by detecting CA mRNA.

17. The method of claim 13, wherein the expression of carbonic anhydrase (CA) is measured by detecting CA polypeptide.
